(19) United States
(12) Patent Application Publication
(10) Pub. No.: US 2011/0200577 A1
(43) Pub. Date: Aug. 18, 2011

(54) BIOMATRICES TO ATTRACT AND RETAIN REGENERATIVE AND REPARATIVE CELLS

(75) Inventors: Stephen L. Smith, Arlington Heights, IL (US); Loredana Campo, Elmhurst, IL (US); Jennifer Campbell, Lake Havasu City, AZ (US); Shiri Uriel Wallach, Glencoe, IL (US); Wanda Seyton, St. Louis, MO (US); Shane Donovan, Lake Geneva, WI (US)

(73) Assignees: BAXTER INTERNATIONAL INC., Deerfield, IL (US); BAXTER HEALTHCARE S.A., Glattpark (Opfikon) (CH)

(21) Appl. No.: 12/987,531

(22) Filed: Jan. 10, 2011

Related U.S. Application Data
(60) Provisional application No. 61/293,525, filed on Jan. 8, 2010.

(51) Int. Cl. A61K 38/48; A61P 43/00

(52) U.S. Cl. 424/94.64

(57) ABSTRACT

The invention relates to a pharmaceutical composition which comprises a fibrin clot and a cytokine and methods of delivering the composition to a site of disease or injury in vivo to attract and retain regenerative or reparative stem or myeloid cells and their differentiated progeny.

### FIELD OF THE INVENTION

The invention generally relates to a pharmaceutical composition which comprises a fibrin biomatrix or a fibrin clot and a cytokine and methods of delivering the composition to a site of disease or injury in vivo to attract and retain regenerative and or reparative stem and myeloid cells and their differentiated progeny.

### BACKGROUND OF THE INVENTION

Stem cells or cells with regenerative capacity have tremendous potential to treat human disease by regenerating or repairing tissue in vivo. Adult stem cells have been used for many years to treat bone marrow failure following irradiation or chemotherapy, as well as a number of congenital hematopoietic conditions. Typically, bone marrow transplants are either autologous (using a patients own stem cells) or allogeneic (using a sibling's or close relative's stem cells). Under certain physiologic or experimental conditions, stem cells can be induced to become cells with specialized functions, such as blood cells or the rhythmic beating cells of heart muscle; hence their utility in treating human heart disease.

Clinical trials are currently underway to treat ischemic heart disease using isolated CD34+ stem cells (CD34 is a cell surface glycoprotein) collected from granulocyte-colony stimulating factor (G-CSF)-mobilized peripheral blood (Losordo et al., Circulation 115:3165-72, 2007). Antibody selection technology (Isolex 300i, Baxter Healthcare Corp., Deerfield, Ill.) is used to harvest the CD34+ expressing cells from the patient's blood. The CD34+ cells are then stored for a period of time and injected using a catheter device into a zone of ischemic heart muscle. Pre-clinical experiments in different animal models of ischemia have shown that these cells have clinical utility in treating ischemic disease, specifically, ischemia induced by coronary heart disease and peripheral vascular disease (Taylor et al., Diabetes Obes. Metab. 10 (Suppl. 4): 5-15, 2008; and Li et al., Thromb. Haemost. 95: 301-11, 2006).

Cell therapy for treating disease is technically and commercially challenging. First, the cells must be collected ex vivo in sufficient quantity to be clinically effective for the treatment. Typically, this would involve autologous cell collection before being re-injected back into the recipient patient. During this process, the cells must be maintained in a viable form for a period of time, retain their regenerative potential, and must not undergo changes during storage that would elicit adverse reactions in patients. Additionally, the cells must be physically delivered to the site of disease or injury in vivo using a catheter or other delivery device. Furthermore, cell collection at individual sites requires site-specific regulatory procedures that negatively impact the commercial viability of this approach.

CD34+ cells are a heterogeneous population, predominantly composed of immature myeloid cells but, also, some immature B cells. There is accumulating evidence that CD34+ cells might not act as stem cells in treatment of ischemic disease. For example, it is now clear that CD34+ cells do not replace damaged muscle cells and neither do they incorporate directly into nascent blood vessels. Rather CD34+ cells act in a paracrine manner to stimulate angiogenesis or enhance vascularization by releasing soluble pro-angiogenic factors (Hofmann et al., Circulation 111: 2198-2202, 2005).

Purified CD34+ cells predominantly exhibit a blast phenotype (90%). Approximately, 33% of CD34+ cells co-express the markers CD45 and Cd11b. When cultured in vitro, these CD34+ cells lose expression of the CD34+ marker, and increase expression of both CD45 and CD11b as a function of time. Coincident with this change in cell surface marker expression is a change from a more immature blast phenotype to a more differentiated myeloid cell type, for example, myelocytes and promyelocytes Using in vitro assays that examine the ability of these cells to support endothelial tube formation—a precursor to the generation of blood vessels both myeloid populations demonstrate pro-angiogenic activity. These experiments indicate that expression of the CD34+ integrin is not an obligate requirement for cells to be pro-angiogenic.

The de novo generation of blood vessels has been carefully examined using genetic approaches in mice. Specifically, experiments have shown that one important component of neo-vascularization is the recruitment of circulating bone marrow-derived mononuclear cells that co-express CD45 and Cd11b markers. These experiments also have shown that the localized expression of stromal cell derived factor-1 (SDF-1) at sites of neovascularization is essential, both to retain these reparative cells in the vicinity of nascent blood vessels and to support neo-vascularization. The cells can be recovered from the site of growing blood vessels and have been shown to stimulate angiogenesis in vitro by secretion of soluble factors that are pro-angiogenic (Grunwald et al., Cell 124: 175-89, 2006).

There is a need in the art for new compositions and methods that stimulate neovascularization. Here we describe a composition comprising fibrin clot and SDF-1 and methods for reparative cell therapy that use the composition to recruit endogenous reparative myeloid cells and their differentiated progeny to a specific site in vivo. The following disclosure describes the specifics of such a composition and methods.

### SUMMARY OF THE INVENTION

The invention addresses one or more needs in the art relating to pharmaceutical compositions which comprise fibrin clot and a cytokine or a combination of cytokines, methods for preparing the composition, and methods of delivering the composition to a site of disease or injury in vivo to attract and retain reparative myeloid cells and their differentiated progeny. In a particular aspect, the cytokine is SDF-1. In other aspects, the cytokine is stem cell factor (SCF). In further aspects, the clot comprises a combination of SDF-1 and SCF.

In one embodiment, the invention includes compositions comprising a fibrin clot and a cytokine or a combination of cytokines. In some aspects, the cytokine is selected from the group consisting of stromal derived factor-1 (SDF-1) and stem cell factor (SCF). In one aspect, the cytokine is SDF-1. In other aspects, the cytokine is SCF. In further aspects, the combination of cytokines comprises stromal derived factor-1 (SDF-1) and stem cell factor (SCF). In some aspects, the fibrin clot comprises any fibrin-based hemostat or sealant. In particular aspects, the fibrin clot is Tisseel® or Tisseel® VHSD or Floseal®. In some aspects, the fibrin clot comprises fibrinogen at a final concentration from about 1 mg/ml to about 100 mg/ml and thrombin at a final concentration from about 0.5 IU/ml to about 250 IU/ml. In other aspects, the fibrin clot comprises fibrinogen at a final concentration of about 10 mg/ml and thrombin at a final concentration of about 2 IU/ml. In further aspects, the compositions of the invention further comprise phosphate buffer or a phosphate-buffered saline solution. In some aspects, the composition comprises SDF-1 at a final concentration from about 1.0 ng/ml to about 50,000 ng/ml. In other aspects, the composition comprises SDF-1 at a final concentration from about 10 ng/ml to about 5,000 ng/ml. In some aspects, the composition comprises SCF at a final concentration from about 1.0 ng/ml to about 50,000 ng/ml. In other aspects, the composition comprises SCF at a final concentration from about 10 ng/ml to about 5,000 ng/ml.

In another embodiment, the invention includes methods of preparing the compositions comprising a fibrin clot and a cytokine or a combination of cytokines. In certain aspects, such methods comprise the step of mixing the cytokine or combination of cytokines with thrombin and adding the cytokine or combination of cytokines and thrombin to fibrinogen to form the fibrin clot composition. In some aspects, the cytokine is selected from the group consisting of stromal derived factor-1 (SDF-1) and stem cell factor (SCF).

In another embodiment, the invention includes methods for recruiting and retaining reparative cells to a localized site of injury or disease in a subject in need thereof. Such methods comprise the step of delivering any of the compositions described herein to the site of injury or disease in an amount effective to recruit and retain reparative cells to the site of injury. In some aspects, the reparative cells are stem cells. In other aspects, the reparative cells are myeloid cells and their differentiated progeny. In particular aspects, the reparative cells are positive for CD34 (CD34+), CD45 (CD45+), or CD11b (CD11b+). In certain aspects the reparative cells are positive for one or more of CD34 (CD34+), CD45 (CD45+), and CD11b (CD11b+).

In another embodiment, the invention includes methods for treating a localized site of injury or disease in a subject in need thereof. Such methods comprise the step of delivering any of the compositions described herein to the site of injury or disease in an amount effective for treating the injury or disease.

In another embodiment, the invention includes methods for enhancing vascularization or inducing angiogenesis to a localized site of injury or disease in a subject in need thereof. Such methods comprise the step of delivering any of the compositions described herein to the site of injury or disease in an amount effective for enhancing vascularization or inducing angiogenesis.

In another embodiment, the invention includes methods of treating ischemia in a subject. Such methods comprise the step of delivering any of the compositions described herein to a site of ischemia in the subject in an amount effective to treat ischemia.

In another embodiment, the invention includes kits for preparing any of the compositions described herein. Such kits comprise a first vial or first storage container comprising fibrinogen; a second vial or second storage container comprising thrombin; and a third vial or third storage container comprising a cytokine or a combination of cytokines, said kit optionally containing a phosphate buffer and instructions for use thereof.

In other embodiments, the invention includes uses of the compositions described herein in the production of a medicament. In some aspects, the invention includes uses of the compositions described herein in the production of a medicament for recruiting and retaining reparative cells to a localized site of injury or disease. In other aspects, the invention includes uses of the compositions described herein in the production of a medicament to treat a localized site of injury or disease. In some aspects, the invention includes uses of the compositions described herein in the production of a medicament for enhancing vascularization or inducing angiogenesis at a localized site of injury or disease. In some aspects, the invention includes uses of the compositions described herein in the production of a medicament for treating ischemia.

Although fibrin-based biomatrices are specifically described in various embodiments of the invention, other biological matrices such as collagen, gelatin, synthetic hydrogels, and the like are included in the invention.

Other features and advantages of the invention will become apparent from the following detailed description. It should be understood, however, that the detailed description and the specific examples, while indicating specific embodiments of the invention, are given by way of illustration only, because various changes and modifications within the spirit and scope of the invention will become apparent to those skilled in the art from this detailed description.

### BRIEF DESCRIPTION OF THE DRAWINGS

FIG. 1 shows binding sensograms of both isoforms of SDF1 [SDF-1α (FIG. 1a) and SDF-1β (FIG. 1b)] to the fibrinogen sealant component of Tisseel®VHSD.

FIG. 2 shows the cumulative release of SDF-1α from Tisseel® VHSD fibrin clots over a period of seven days.

FIG. 3a depicts freshly isolated CD34+ myeloid cells in Matrigel® (BD Biosciences). The Matrigel® assay showed that at a 7-day time point, endothelial cell tubes did not regress when CD34+ cells were present when compared to endothelial cells alone. FIG. 3b shows that myeloid cells that co-express CD45b and CD11b are pro-angiogenic in vitro.

FIG. 4 a shows the recruitment of CD45+/CD11b+ myeloid cells to Tisseel®-SDF-1 clots implanted in vivo. Cells that co-express CD45+ and CD11b were recruited to Tisseel®-SDF-1 clots in greater numbers than to Tisseel® clots without SDF-1. FIG. 4b shows that SDF-1 increased the in vivo recruitment of EGFP+ and CD45+/CD11b+ bone marrow-derived cells to intramuscularly injected Tisseel®-SDF-1 clots compared to controls (PBS). FIG. 4c shows the recruitment of stem cell antigen-1 (Sca-1)+ cells in vivo. Tisseel®-SDF-1 clots implanted in vivo recruited greater numbers of cells that express Sca1, the murine equivalent of CD34.

### DETAILED DESCRIPTION OF THE INVENTION

The invention provides a biodegradable, biocompatible fibrin biomatrix or fibrin clot which is used to deliver various cytokines including, without limitation, SDF-1 and/or SCF. In certain aspects, the invention provides various formulations of fibrin clot in which to deliver cytokines to recruit reparative cells to a site of disease or injury and increase retention time of the cells at the site. The fibrin clot provides a three-dimensional matrix to deliver a cytokine, recruit reparative cells and mimic an in vivo environment in tissues or organs. The present invention provides such formulations of fibrin clots and methods for their use.

Before any embodiments of the invention are explained in detail, it is to be understood that the invention is not limited in its application to the details of construction and the arrangement of components set forth in the following description or illustrated in the figures and examples. The section headings used herein are for organizational purposes only and are not to be construed as limiting the subject matter described. All references cited in this application are expressly incorporated by reference herein.

The invention embraces other embodiments and is practiced or carried out in various ways. Also, it is to be understood that the phraseology and terminology used herein is for the purpose of description and should not be regarded as limiting. The terms “including,” “comprising,” or “having” and variations thereof are meant to encompass the items listed thereafter and equivalents thereof as well as additional items.

### DEFINITIONS

Unless otherwise defined, all technical and scientific terms used herein have the same meaning as commonly understood by one of ordinary skill in the art to which this invention belongs. The following references provide one of skill with a general definition of many of the terms used in this invention: Singleton, et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger, et al. (eds.), Springer Verlag (1991); and Hale and Marham, THE HARPER COLLINS DICTIONARY OF BIOLOGY (1991).

The following abbreviations are used throughout.

EGFP Enhanced green fluorescent protein
FACS Fluorescence activated cell sorting
GM-CSF Granulocyte-macrophage colony-stimulating factor
SDF-1 Stromal derived factor-1
SDF-1α Stromal derived factor-1 alpha
SDF-1β Stromal derived factor-1 beta
HUVEC Human umbilical vein endothelial cell
IU International units

### kDa KiloDaltons

### μl or μL Microliter

### ml or mL Milliliter

### ng Nanogram

It is noted here that, as used in this specification and the appended claims, the singular forms “a,” “an,” and “the” include plural reference unless the context clearly dictates otherwise.

The terms “fibrin,” “fibrin matrix,” “fibrin biomatrix,” “fibrin clot,” “fibrin-based scaffold,” “fibrin scaffold,” “fibrin glue,” “fibrin gel,” “fibrin adhesive,” and “fibrin sealant” are often used interchangeably herein and in the art to refer to a three-dimensional network comprising at least a fibrinogen component and a thrombin component. The invention includes fibrin clots comprising any fibrin-based hemostat or sealant as described herein.

The term “cytokine” refers to a diverse group of small secreted proteins which play a critical role in tissue regeneration and in the mediation and regulation of immunity, inflammation, and hematopoiesis. The term “combination of cytokines” refers to any two or more cytokines known in the art including, without limitation, stromal cell derived factor-1 (SDF-1) and stem cell factor (SCF).

The term “SDF-1” or “SDF-1α” or “SDF-1β” refers to stromal cell derived factor-1 polypeptide, a small cytokine belonging to the chemokine family. In various aspects, the terms “SDF-1” and “SDF-1α” are used interchangeably. In other aspects, the terms “SDF-1” and “SDF-1β” are used interchangeably.

The term “SCF” refers to stem cell factor polypeptide, a cytokine belonging to the chemokine family.

The terms “polypeptide,” “peptide” and “protein” are used interchangeably herein to refer to a polymer of amino acid residues linked via peptide bonds. The terms apply to amino acid polymers in which one or more amino acid residue is an artificial chemical mimetic of a corresponding naturally occurring amino acid, as well as to naturally occurring amino acid polymers and non-naturally occurring amino acid polymers. The term “protein” typically refers to large polypeptides. The term “peptide” typically refers to short polypeptides. Synthetic polypeptides can be prepared, for example, using an automated polypeptide synthesizer.

It is specifically understood that any numerical value range disclosed herein includes all values from the lower value to the upper value, i.e., all possible combinations of numerical values between the lowest value and the highest value enumerated are to be considered to be expressly stated in this application. For example, if a concentration range is stated as about 1% to 50%, it is intended that values such as 2% to 40%, 10% to 30%, or 1% to 3%, etc., are expressly enumerated in this specification. The values listed above are only examples of what is specifically intended.

Ranges, in various aspects, are expressed herein as from “about” or “approximately” one particular value and/or to “about” or “approximately” another particular value. When values are expressed as approximations by use of the antecedent “about,” it will be understood that some amount of variation is included in the range.

As used herein a “fragment” of a polypeptide refers to any portion of the polypeptide less than the full-length polypeptide or protein expression product. Fragments are typically deletion analogs of the full-length polypeptide wherein one or more amino acid residues have been removed from the amino terminus and/or the carboxy terminus of the full-length polypeptide. Accordingly, “fragments” are a subset of deletion analogs described below. The invention includes fragments of polypeptides that retain biological activity. For example, the invention includes biologically active fragments of the cytokines disclosed herein.

As used herein an “analog” refers to a polypeptide substantially similar in structure and having the same or essentially the same biological activity, albeit in certain instances to a differing degree, to a naturally-occurring molecule. Analogs differ in the composition of their amino acid sequences compared to the naturally-occurring polypeptide from which the analog is derived, based on one or more mutations involving (i) deletion of one or more amino acid residues at one or more termini of the polypeptide (including fragments as described above) and/or one or more internal regions of the naturally-occurring polypeptide sequence, (ii) insertion or addition of one or more amino acids at one or more termini (typically an “addition” analog) of the polypeptide and/or one or more internal regions (typically an “insertion” analog) of the naturally-occurring polypeptide sequence or (iii) substitution of one or more amino acids for other amino acids in the naturally-occurring polypeptide sequence. Substitutions are conservative or non-conservative based on the physico-chemical or functional relatedness of the amino acid that is being replaced and the amino acid replacing it. The invention includes analogs of the cytokine polypeptides disclosed herein.

A “conservatively modified analog” is a polypeptide comprising conservative substitutions of amino acids with chemically similar amino acids. Conservative substitution tables providing functionally similar amino acids are well known in the art. The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

8) Cysteine (C), Methionine (M) (see, e.g., Creighton, Proteins (1984)).

Such conservatively modified analogs are in addition to and do not exclude polymorphic variants, interspecies homologs, and allelic variants.

An “allelic variant” typically refers to any of two or more polymorphic forms of a gene occupying the same genetic locus. Allelic variations arise naturally through mutation, and may result in phenotypic polymorphism within populations. In certain aspects, gene mutations are silent (no change in the encoded polypeptide) or may encode polypeptides having altered amino acid sequences. “Allelic variants” also refer to cDNAs derived from mRNA transcripts of genetic allelic variants, as well as the proteins encoded by them.

As used herein a “variant” refers to a polypeptide, protein or analog thereof that is modified to comprise additional chemical moieties not normally a part of the molecule. Such moieties, in various aspects, modulate the molecule's solubility, absorption, and/or biological half-life. The moieties, in various other aspects, alternatively decrease the toxicity of the molecule and eliminate or attenuate any undesirable side effect of the molecule, etc. Moieties capable of mediating such effects are disclosed in Remington's Pharmaceutical Sciences (1980). Procedure for coupling such moieties to a molecule are well known in the art. For example, the variant, in some aspects, is a cytokine polypeptide molecule having a chemical modification which confers a longer half-life in vivo to the cytokine polypeptide. In one embodiment, the polypeptides are modified by addition of a water soluble polymer known in the art. In a related embodiment, polypeptides are modified by glycosylation, PEGylation, and/or polysialylation. The invention includes variants of the cytokine polypeptides disclosed herein.

As used herein “selectable marker” refers to a gene encoding an enzyme or other protein that confers upon the cell or organism in which it is expressed an identifiable phenotypic change such as resistance to a drug, antibiotic or other agent, such that expression or activity of the marker is selected for (for example, but without limitation, a positive marker, such as the neo gene) or against (for example, and without limitation, a negative marker, such as the diphtheria gene). A “heterologous selectable marker” refers to a selectable marker gene that has been inserted into the genome of an animal in which it would not normally be found.

Examples of selectable markers include, but are not limited to, an antibiotic resistance gene such as neomycin (neo), puromycin (Puro), diphtheria toxin, phosphotransferase, hygromycin phosphotransferase, xanthineguanine phosphoribosyl transferase, the Herpes simplex virus type 1 thymidine kinase, adenine phosphoribosyltransferase and hypoxanthine phosphonbosyltransferase. In one aspect of the invention, a selectable marker is the enhanced green fluorescent protein (EGFP). EGFP is suitable as a control reagent for expression studies and is used herein as described in the Examples. The worker of ordinary skill in the art will understand any selectable marker known in the art is useful in the methods described herein.

The term “agent” or “compound” describes any molecule, e.g. protein or pharmaceutical, with the capability of affecting a biological parameter in the invention.

A “control,” as used herein, can refer to an active, positive, negative or vehicle control. As will be understood by those of skill in the art, controls are used to establish the relevance of experimental results, and provide a comparison for the condition being tested.

The terms “pharmaceutically acceptable carrier” or “physiologically acceptable carrier” as used herein refer to one or more formulation materials suitable for accomplishing or enhancing the delivery of a composition of the invention.

The terms “effective amount,” “amount effective,” and “therapeutically effective amount” each refer to the amount of a composition comprising fibrin clot and cytokine to achieve an observable change in a subject as set forth herein. For example, in certain aspects of the invention, an effective amount would be the amount necessary to attract and retain stem cells or proangiogenic myeloid cells and their differentiated progeny to the fibrin clot in vivo. An “effective amount,” “amount effective,” and “therapeutically effective amount” of a composition to be employed therapeutically will depend, for example, upon the therapeutic context and objectives.

The term “combination” refers to one or more polypeptides or compositions of the invention. In some aspects, combinations of molecules of the invention are administered to provide increased angiogenesis or enhanced vascularization at a site of injury or disease.

A “subject” is given its conventional meaning of a non-plant, non-protist living being. In most aspects, the subject is an animal. In particular aspects, the animal is a mammal. In more particular aspects, the mammal is a human. In other aspects, the mammal is a pet or companion animal, a domesticated farm animal, or a zoo animal. In certain aspects, the mammal is a cat, dog, horse, or cow.

Fibrin Clot

In some aspects, the invention includes fibrin clots formed by mixing the fibrinogen component with the thrombin component. Fibrin, also known as factor Ia, is a fibrous, non-globular protein involved in the clotting of blood. More specifically, fibrin is produced from cleavage of fibrinogen, a soluble plasma glycoprotein that is synthesized by the liver and found in blood plasma. Processes in the coagulation cascade activate the zymogen prothrombin to the serine protease thrombin, which is responsible for converting fibrinogen into fibrin. Fibrin molecules then combine to form long fibrin threads that entangle platelets, building up a spongy mass that gradually forms a complex polymer which contracts to form the blood clot. This hardening process is stabilized by a substance known as fibrin-stabilizing factor, or factor XIII.

A fibrin clot or fibrin matrix is a network of protein that holds together and supports a variety of living tissues, especially in response to injury. This fibrin clot exploits the final stage of the coagulation cascade in which fibrinogen molecules are cleaved by thrombin, convert into fibrin monomers and assemble into fibrils, eventually forming fibers in a three-dimensional network. Simultaneously, factor XIII (FXIII) present in the solution is activated by thrombin in the presence of calcium ions to factor XIIIa. The aggregated fibrin monomers and any remaining fibronectin possibly present are cross-linked to form a high polymer by new peptide bonds forming. By this cross-linking reaction, the strength of the clot formed is substantially increased. Generally, the clot adheres well to wound and tissue surfaces, which leads to the adhesive and haemostatic effect. (See U.S. Pat. No. 7,241,603). Therefore, fibrin adhesives are frequently used as two-component adhesives which comprise a fibrinogen complex component together with a thrombin component which additionally contains calcium ions.

In the invention, the fibrin clot is a three-dimensional network comprising at least a fibrinogen component and a thrombin component, which can act as a scaffold for delivery of a cytokine or a combination of cytokines over time. In some aspects, the cytokine is any cytokine that is involved in hematopiesis and stem cell proliferation. In some aspects, the cytokine is SDF-1. In other aspects, the cytokine is SCF. In more particular aspects, the fibrin clot comprises a combination of cytokines. In particular aspects, the invention includes a combination of SDF-1 and SCF.

Such fibrin matrix or fibrin clot is provided naturally by the body after injury, but also can be engineered as a tissue substitute as described herein to speed healing. The fibrin clot consists of naturally occurring biomaterials composed of cross-linked fibrin network and has a broad use in biomedical applications. For example, it is used to control surgical bleeding, speed wound healing, seal off hollow body organs or cover holes made by standard sutures, and provide slow-release delivery of medications like antibiotics to tissues exposed. Such a fibrin clot is useful in repairing injuries to the body, and is useful in sites of ischemia. In biomedical research, fibrin clots have been used to fill bone cavities, and repair neurons, heart valves and the surface of the eye. Fibrin clots have also been used in the urinary tract, liver, lung, spleen, kidney, and hear. In the present invention, fibrin clots are used in any site of the body.

Fibrin sealants are a type of surgical tissue adhesive derived from human and animal blood products. The ingredients in fibrin sealants interact during application to form a stable clot composed of fibrin. Fibrin sealants are used to control surgical bleeding, speed wound healing, seal off hollow body organs or cover holes made by standard sutures, and provide slow-release delivery of medications like antibiotics to exposed tissues. As of about 2003, all fibrin sealants used in the United States are made from blood plasma taken from carefully screened donors and rigorously tested to eliminate hepatitis viruses, HIV-1, and parvovirus. All fibrin sealants in use as of 2003 have two major ingredients, purified fibrinogen protein and purified thrombin enzyme derived from human or bovine (cattle) blood. Many sealants have two additional ingredients, human blood factor XIII and aprotinin, which is derived from cows' lungs. Factor XIII strengthens blood clots by forming cross-links between strands of fibrin. Aprotinin inhibits the enzymes that break down blood clots. Examples of fibrin sealants are described in U.S. Pat. Nos. 5,716,645; 5,962,405; and 6,579,537 and are available in lyophilized, frozen, or non-frozen liquid form. Fibrin sealants have also been designed which lack the aprotinin ingredient (EVICEL, Ethicon, Inc., New Jersey). The invention includes the use of all types of fibrin sealants.

A particular advantage of a fibrin sealant is that the adhesive/gel does not remain at its site of application as a foreign body, but is completely resorbed just as in natural wound healing, and is replaced by newly formed tissue. Various cells, e.g., macrophages and, subsequently, fibroblasts migrate into the gel, lyse, and resorb the gel material and form new tissue. Fibrin sealants have been used to form fibrin clots or fibrin gels in situ, and these fibrin gels have been used for delivery of cells and growth factors (Cox et al., Tissue Eng. 10:942-954, 2004; and Wong et al., Thromb. Haemost. 89:573-582, 2003).

In some aspects of the invention, fibrin sealants such as Tisseel® (Baxter International Inc.) and Tisseel® Vapor Heat Solvent Detergent (Tisseel® VHSD) (Baxter International Inc.), a next generation fibrin sealant, are used. Tisseel® VHSD was developed with an added virus inactivation step (solvent/detergent [S/D] treatment) to provide added safety and convenience to the currently licensed Tisseel® product. Tisseel® VHSD is indicated for use as an adjunct to hemostasis in surgeries involving cardiopulmonary bypass and treatment of splenic injuries. In particular aspects of the invention, a fibrin clot is prepared from Tisseel® or Tisseel® VHSD. In other aspects, fibrin sealants such as Floseal® (Baxter International Inc.) are used. Floseal® is an effective hemostatatic matrix that stops bleeding in 2 minutes or less (median time to hemostasis).

As discussed herein above, fibrin clots are, in one aspect, prepared from separate solutions of thrombin and fibrinogen. The thrombin and fibrinogen solutions are loaded into a double-barreled syringe that allows them to mix and combine. As the thrombin and fibrinogen solutions combine, a clot develops in the same way that it would form during normal blood clotting through a series of chemical reactions known as the coagulation cascade. At the end of the cascade, thrombin breaks up fibrinogen molecules into fibrin molecules that arrange into strands that are then cross-linked by Factor XIII to form a lattice or net-like pattern that stabilizes the clot.

Additional methods for producing fibrinogen-containing preparations that can be used as tissue adhesives include production from cryoprecipitate, optionally with further washing and precipitation steps with ethanol, ammonium sulphate, polyethylene glycol, glycine or beta-alanine, and production from plasma within the scope of the known plasma fractionation methods, respectively (cf., e.g., “Methods of plasma protein fractionation”, 1980, ed.: Curling, Academic Press, pp. 3-15, 33-36 and 57-74, or Blomb ck B. and M., “Purification of human and bovine fibrinogen”, Arkiv. Kemi. 10, 1959, p. 415 f.). Fibrin clots or fibrin sealants, in some aspects, are made using a patient's own blood plasma. For example, the CRYOSEAL (Thermogenesis Corp., Rancho Cordova, Calif.) or VIVOSTAT (Vivolution A/S, Denmark) fibrin sealant systems enable the production of autologous fibrin sealant components from a patient's blood plasma. The components of fibrin sealants are available in lyophilized, deep-frozen liquid, or liquid form.

As discussed above, in various aspects, the fibrin clot comprises fibrinogen and thrombin. Polymerization time of fibrinogen and thrombin is affected by both the concentration of fibrinogen and thrombin as well as by temperature. Fibrin clot characterization by scanning electron microscopy reveals that thick fibers make up a dense structure at lower fibrinogen concentrations and thinner fibers and a tighter gel can be obtained as fibrinogen concentration increases. In certain aspects, fibrin clot structure is modified by the dilution buffer used in preparing the fibrin clot. Thrombin concentration does not appear to affect polymerization as greatly as fibrinogen, but under defined fibrinogen concentrations, the fiber clot fibers steadily get thinner with increasing concentrations of thrombin. In further aspects, the fibrin clot also comprises collagen, fibronectin, and other matrix proteins. In additional aspects, the fibrin clot is bioabsorbable and biocompatible.

Other Biological Biomatrices

Although fibrin-based biomatrices are specifically described in various embodiments of the invention, other biological matrices such as collagen, gelatin, synthetic hydrogels, and the like are included in the invention. In various aspects, like the fibrin-based biomatrices described herein, these other biological matrices comprise a cytokine or a combination of cytokines. In particular aspects, these biomatrices comprise SDF-1, SCF, or a combination of SDF-1 and SCF.

Cytokines

The invention includes compositions and methods comprising one or more cytokines. The term “cytokine” is a generic name for a diverse group of small secreted proteins which play a critical role in tissue regeneration and in the mediation and regulation of immunity, inflammation, and hematopoiesis. Although “cytokine” is a general name used for these small secreted proteins, other names include lymphokine (cytokines made by lymphocytes), monokine (cytokines made by monocytes), chemokine (cytokines with chemotactic activities), and interleukin (cytokines made by one leukocyte and acting on other leukocytes), and all are included for use in the invention. In particular aspects, and without limitation, cytokines include activin, bone morphogenic protein (BMP), epidermal growth factor (EGF), fibroblast growth factor (FGF), Flt-3/Flk-2 ligand, granulocyte colony stimulating factor (G-CSF), granulocyte-macrophage colony stimulating factor (GM-CSF), insulin like growth factor-1 and -2 (IGF-1 and IGF-2), interferon-γ (IFN-γ), interleukin-1 to -15 (IL-1 through IL-15), macrophage colony stimulating factor (M-CSF), neurotrophin, platelet-derived growth factor-AB (PDGF-AB), stem cell factor (SCF), stromal cell-derived factor-1 (SDF-1), transforming growth factor-β (TGF-β), tumor necrosis factor-α (TNF-α), and vascular endothelial growth factor (VEGF).

Stromal Derived Factor-1 (SDF-1)

In some aspects, the invention includes stromal cell-derived factor-1 (SDF-1) polypeptide molecules, fragments and analogs thereof, and compositions comprising these molecules. SDF-1 is small cytokine belonging to the chemokine family that is officially designated Chemokine (C-X-C motif) ligand 12 (CXCL12). SDF-1 is produced in two forms, SDF-1α/CXCL12a (SDF-1α) and SDF-1β/CXCL12b (SDF-1β), by alternate splicing of the same gene. In various aspects, both forms, SDF-1α and SDF-1β, are used, and the term “SDF-1” is used interchangeably herein to refer to one or both forms.

Chemokines are characterized by the presence of four conserved cysteines, which form two disulfide bonds. The CXCL12 proteins belong to the group of CXC chemokines, whose initial pair of cysteines are separated by one intervening amino acid. CXCL12 plays an important role in angiogenesis by recruiting endothelial progenitor cells (EPCs) from the bone morrow through a CXCR4-dependent mechanism.

In certain aspects of the invention, SDF-1 is added to the fibrinogen component prior to mixing with the thrombin component or after the clot has been formed. The fibrin clot containing SDF-1 subsequently releases SDF-1 from the clot into the environment. The released SDF-1 in turn attracts cells to the site where the clot was applied. The relatively high local concentration of SDF-1 within the fibrin clot helps retain the cells in the vicinity of the clot. In various aspects, this preparation is applied at many different sites in patients, as Tisseel® VHSD is already used in a wide number of different surgical applications where it displays broad tissue adhesiveness and compatibility.

Stem Cell Factor (SCF)

In some aspects, the invention includes stem cell factor (SCF), also known as kit-ligand, KL, or steel factor, polypeptide molecules, fragments and analogs thereof, and compositions comprising these molecules. SCF is a cytokine that binds to the c-Kit receptor (CD117). SCF can exist both as a soluble protein and as a transmembrane protein, and both forms are required for normal hematopoietic function. In various aspects, both forms of SCF are used.

SCF plays an important role in hematopoiesis (formation of blood cells), spermatogenesis, and melanogenesis. In a particular aspect, SCF plays an important role in the hematopoiesis during embryonic development. Sites where hematopoiesis takes place, such as the fetal liver and bone marrow, all express SCF. Without being bound by theory, SCF may serve to direct hematopoietic stem cells (HSCs) to their stem cell niche (the microenvironment in which a stem cell resides), and it plays an important role in HSC maintenance.

In certain aspects of the invention, SCF is added to the fibrinogen component prior to mixing with the thrombin component or after the clot has been formed. The fibrin clot containing SCF subsequently releases SCF from the gel into the environment. The released SCF in turn attracts cells to the site where the gel was applied. The relatively high local concentration of SCF within the fibrin clot helps retain the cells in the vicinity of the clot. In various aspects, this preparation is applied at many different sites in patients, as Tisseel® VHSD is already used in a wide number of different surgical applications where it displays broad tissue adhesiveness and compatibility.

Cells

In some aspects, the invention includes methods of using fibrin clot comprising a cytokine to recruit reparative or regenerative cells to a site of injury or disease in vivo. In certain aspects of the invention, the reparative cell is a stem cell. A stem cell is a cell that has the potential to regenerate tissue over a lifetime. For example, the gold standard test for a bone marrow (BM) or hematopoietic stem cell (HSC) is the ability to transplant one cell and save an individual without HSCs. In some aspects of the invention, the reparative cell is a myeloid cell. The myeloid cell line begins as myeloid stem cells produced in the BM. Myeloid cells, in various aspects, mature into several types of blood cells, including megakaryocytes, erythrocytes, macrophages, eosinophils, neutrophils, and basophils. In further aspects, the invention includes differentiated progeny of myeloid cells.

Myeloid Cell-Induced Angiogenesis

In certain aspects, the invention includes methods of using a composition comprising fibrin clot and a cytokine to recruit myeloid cells and their differentiated progeny in vivo to induce angiogenesis or enhance vascularization. In the adult, angiogenesis is essential to wound repair and inflammation and for highly specialized functions, such as the regeneration of the endometrium. One of the important findings over the past few years has been the identification of the role of different cell types in angiogenesis.

As with endothelial cells (ECs) and their BM precursors, heterogeneous populations of BM-derived cells of the myeloid lineage contribute to the angiogenic process. These cell populations, originating from a common BM-derived precursor, circulate in peripheral blood until recruited to tissues by specific chemoattractants.

In various aspects, stem cells are recruited to the fibrin clot comprising the cytokine. In a particular aspect, cells expressing CD34 (CD34+ cells) are recruited. CD34+ cells are a heterogeneous population, predominantly composed of immature myeloid cells but, also, some immature B cells. There is accumulating evidence that CD34+ cells might not act as stem cells in treatment of ischemic disease. Purified CD34+ cells predominantly exhibit a blast phenotype (90%). Approximately, 33% of CD34+ cells co-express the markers CD45 and Cd11b. Thus, in various aspects, cells expressing CD45 (CD45+ cells) and/or cells expressing CD11b (CD11b+ cells) are recruited. CD11b+ cells are myeloid progenitor cells, originating from the bone marrow, and are involved in promoting angiogenesis and lymphangiogenesis. In yet a further aspect of the invention, cells expressing one or more, or a combination, of CD34, CD45, and CD11b are recruited to the fibrin clot.

The terms “enhancing vascularization” or “inducing angiogenesis” are used herein to describe means of forming vascular networks. Vasculogenesis and angiogenesis are the fundamental processes by which new blood vessels are formed. In the art, vasculogenesis is defined as the differentiation of precursor cells (angioblasts) into endothelial cells and the de novo formation of a primitive vascular network, whereas angiogenesis is defined as the growth of new capillaries from pre-existing blood vessels. Capillary tube formation or endothelial tube formation represents a specialized endothelial cell function and is a prerequisite for the establishment of a continuous vessel lumen. The invention includes compositions and methods to enhance vascularization as well as induce or enhance angiogenesis.

The de novo generation of blood vessels has been carefully examined using genetic approaches in mice. Specifically, experiments have shown that one important component of neo-vascularization is the recruitment of circulating bone marrow-derived mononuclear cells that co-express CD45 and Cd11b markers.

In other aspects, cells expressing stem cell antigen-1 (Sca-1), the murine equivalent of CD34 are recruited. Sca-1 is a murine cell surface antigen expressed on immature hematopoietic progenitor cells and, together with other markers, defines hematopoietic stem cells. The CD34 protein is a member of a family of single-pass transmembrane sialomucin proteins that show expression on early hematopoietic and vascular-associated tissue.

Compositions and Methods

Aspects of the invention provide compositions and methods for regenerative medicine. In one aspect, the invention provides for compositions comprising biocompatible matrix or fibrin clot materials and cytokines. In certain aspects, the cytokine is SDF-1. In other aspects, the cytokine is SCF.

The components of the fibrin clot are added at appropriate concentrations to provide the type of controlled release desired. Fibrinogen is added in varying concentrations including, but not limited to, about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, about 25 mg/ml, about 26 mg/ml, about 27 mg/ml, about 28 mg/ml, about 29 mg/ml, about 30 mg/ml, about 31 mg/ml, about 32 mg/ml, about 33 mg/ml, about 34 mg/ml, about 35 mg/ml, about 36 mg/ml, about 37 mg/ml, about 38 mg/ml, about 39 mg/ml, about 40 mg/ml, about 41 mg/ml, about 42 mg/ml, about 43 mg/ml, about 44 mg/ml, about 45 mg/ml, about 46 mg/ml, about 47 mg/ml, about 48 mg/ml, about 49 mg/ml, about 50 mg/ml, about 60 mg/ml, about 70 mg/ml, about 80 mg/ml, about 90 mg/ml, about 100 mg/ml, about 150 mg/ml, and up to about 200 mg/ml (final concentrations in the gels), or in intermediate concentrations as necessary. In certain aspects, the fibrinogen is added at concentrations of about 10 mg/ml and about 20 mg/ml.

Further, the fibrinogen may be combined with any appropriate concentration of thrombin. Thrombin is added in varying concentrations including, but not limited to, about 0.1 IU/ml, about 0.2 IU/ml, about 0.3 IU/ml, about 0.4 IU/ml, about 0.5 IU/ml, about 0.6 IU/ml, about 0.7 IU/ml, about 0.8 IU/ml, about 0.9 IU/ml, about 1 IU/ml, about 1.1 IU/ml, about 1.2 IU/ml, about 1.3 IU/ml, about 1.4 IU/ml, about 1.5 IU/ml, about 2 IU/ml, about 3 IU/ml, about 4 IU/ml, about 5 IU/ml, about 6 IU/ml, about 7 IU/ml, about 8 IU/ml, about 9 IU/ml, about 10 IU/ml, about 11 IU/ml, about 12 IU/ml, about 13 IU/ml, about 14 IU/ml, about 15 IU/ml, about 16 IU/ml, about 17 IU/ml, about 18 IU/ml, about 19 IU/ml, about 20 IU/ml, about 21 IU/ml, about 22 IU/ml, about 23 IU/ml, about 24 IU/ml, about 25 IU/ml, about 30 IU/ml, about 35 IU/ml, about 40 IU/ml, about 45 IU/ml, about 50 IU/ml, about 60 IU/ml, about 70 IU/ml, about 80 IU/ml, about 90 IU/ml, about 100 IU/ml, about 110 IU/ml, about 120 IU/ml, about 130 IU/ml, about 140 IU/ml, about 150 IU/ml, about 160 IU/ml, about 170 IU/ml, about 180 IU/ml, about 190 IU/ml, about 200 IU/ml, about 225 IU/ml, about 250 IU/ml, about 275 IU/ml, about 300 IU/ml, or in intermediate concentrations as necessary. In certain aspects, thrombin is added at concentrations of about 2 IU/ml, and about 4 IU/ml.

In some aspects, the compositions of the invention comprise fibrin clot formulations (thrombin to fibrinogen ratios) in ratios ranging from about 0.001 to about 100.0. In another aspect, thrombin to fibrinogen ratios range from about 0.01 to about 10.0. In various aspects, the ratio is about 0.04, or about 0.05, or about 0.11. The invention includes, but is not limited to, the following fibrinogen to thrombin ratios: about 0.001, about 0.005, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, about 10, about 15, about 20, about 25, about 30, about 35, about 40, about 45, about 50, about 55, about 60, about 65, about 70, about 75, about 80, about 85, about 90, about 95, and about 100, or in intermediate ratios as necessary.

In particular aspects of the invention, the fibrin clot comprises fibrinogen at a final concentration from about 1 mg/ml to about 100 mg/ml and thrombin at a final concentration from about 0.5 IU/ml to about 250 IU/ml. In more particular aspects of the invention, fibrin clot formulations (final concentrations of fibrinogen (mg/ml) to thrombin (international units (IU) or units (U)/ml) are about 10 mg/2 U and about 20 mg/4 U.

In various aspects of the invention, synthetic polymers can be mixed with fibrin to form a biodegradable hybrid clot or matrix. Such synthetic polymers include, but are not limited to, polymers such as poly(lactide) (PLA), poly(glycolic acid) (PGA), poly(lactide-co-glycolide) (PLGA). poly(caprolactone), polycarbonates, polyamides, polyanhydrides, polyamino acids, polyortho esters, polyacetals, polycyanoacrylates and degradable polyurethanes, and non-erodible polymers such as polyacrylates, ethylene-vinyl acetate polymers and other acyl substituted cellulose acetates and derivatives thereof.

In one aspect, a composition of the invention comprises fibrin clot and cytokines. In a particular aspect, a composition of the invention comprises fibrin clot and SDF-1. In other aspects, a composition of the invention comprises fibrin clot and SCF. In various aspects, a composition of the invention comprises fibrin clot, SDF-1, and SCF.

In another aspect, the invention provides for delivery of the fibrin clot in vivo for the purpose of recruiting reparative cells to a site of surgery or injury, or a site in the body in need of tissue repair. In one aspect, compositions comprising fibrin clot and a cytokine are administered with the cytokine in a concentration ranging from about 1 ng to about 50,000 ng per volume (1 mL) of fibrin clot. In another aspect, compositions comprising fibrin clot and a cytokine are administered with the cytokine in a concentration ranging from about 10 ng to about 5,000 ng per ml of fibrin clot. In various aspects, the cytokine is administered in the fibrin clot in a concentration of about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 20 ng/ml, about 30 ng/ml, about 40 ng/ml, about 50 ng/ml, about 60 ng/ml, about 70 ng/ml, about 80 ng/ml, about 90 ng/ml, about 100 ng/ml, about 120 ng/ml, about 140 ng/ml, about 160 ng/ml, about 180 ng/ml, about 200 ng/ml, about 300 ng/ml, about 400 ng/ml, about 500 ng/ml, about 600 ng/ml, about 700 ng/ml, about 800 ng/ml, about 900 ng/ml, about 1000 ng/ml, about 1100 ng/ml, about 1200 ng/ml, about 1300 ng/ml, about 1400 ng/ml, about 1500 ng/ml, about 1600 ng/ml, about 1700 ng/ml, about 1800 ng/ml, about 1900 ng/ml, about 2000 ng/ml, about 2500 ng/ml, about 3000 ng/ml, about 3500 ng/ml, about 4000 ng/ml, about 4500 ng/ml, about 5000 ng/ml, about 5500 ng/ml, about 6000 ng/ml, about 6500 ng/ml, about 7000 ng/ml, about 7500 ng/ml, about 8000 ng/ml, about 8500 ng/ml, about 9000 ng/ml, about 9500 ng/ml, about 10,000 ng/ml, about 20,000 ng/ml, about 30,000 ng/ml, about 40,000 ng/ml, and about 50,000 ng/ml. In certain aspects, the cytokine is administered at concentrations of up to about 100 μg/ml and about 1 mg/ml. One skilled in the art will appreciate that the appropriate concentrations of cytokine for treatment will thus vary depending, in part, upon the volume of the fibrin clot, the tissue cite to which the fibrin clot is delivered, the indication for which the fibrin clot is being used, the route of administration, and the size (body weight, body surface or organ size) and condition (the age and general health) of the patient. Accordingly, the clinician may titer the dosage and modify the concentration delivered to obtain the optimal therapeutic effect.

Delivery

In aspects of the invention, the composition is delivered to a patient by several means. In some aspects, the composition is delivered intramuscularly, intraperitoneally, intracranially, between tissue components such as fractured or broken bone or cartilage. In other aspects, the composition is delivered parenterally through injection by intravenous, intracerebral (intraparenchymal), intracerebroventricular, intracerebrospinal, intraocular, intraarterial, intraarticular, intraportal, intrarectal, intranasal, or intralesional routes. In addition, the composition can be introduced for treatment into a mammal by other modes, such as but not limited to, intratumor, topical, subconjunctival, intrabladder, intravaginal, epidural, intracostal, intradermal, inhalation, transdermal, transserosal, intrabuccal, dissolution in the mouth or other body cavities, instillation to the airway, insuflation through the airway, injection into vessels, tumors, organ and the like, and injection or deposition into cavities in the body of a mammal. In a particular aspect, the composition is delivered surgically.

In another aspect, delivery of the composition is targeted to any site in the body. In certain aspects, the target body site is in the nerves, liver, kidney, heart, lung, eye, organs of the gastrointestinal tract, skin, and/or brain. In one aspect, the target body site is a site of ischemia.

The invention includes many various vehicles for delivering the composition into a subject. In one aspect, direct injection by needle and syringe is used. In certain aspects, direct injection includes mixing fibrin clot and cytokine in the syringe immediately prior to injection in a subject. In other aspects, the invention includes the use of a mixing chamber (between syringe and needle) to increase mixing. In various aspects, the invention includes delivery of the cytokinein the fibrin clot via an injection catheter (for deeper tissue delivery), a spray for surface delivery, or by implanting pre-made fibrin clot (subcutaneous or deeper within tissue beds). In certain instances, the implanting can be carried out via injection or via surgery.

Where desired, the composition is administered by bolus injection or continuously by infusion, or by implantation device. Alternatively or additionally, the composition is administered locally via implantation of a membrane, sponge, or another appropriate material on to which the clot has been absorbed or encapsulated. Where an implantation device is used, the device, in various aspects, is implanted into any suitable tissue or organ, and delivery of the composition may be via diffusion, timed release bolus, or continuous administration.

In certain aspects, it may be desirable to use or administer the composition in an ex vivo manner. In such instances, cells, tissues, or organs that have been removed from the patient are exposed to the composition after which the cells, tissues and/or organs are subsequently implanted back into the patient.

In other aspects of the invention, additional ways of delivering the composition to a subject will be evident to those skilled in the art, including formulations involving fibrin clot in sustained or controlled delivery formulations. Techniques for formulating a variety of other sustained or controlled delivery means are known to those skilled in the art.

In one aspect, delivery in a subject is made by injection with a syringe and needle. In a certain aspect, delivery is made with a syringe and 25-gauge needle. However, various sizes of syringes and needles are also used for delivery. In various aspects, the syringe may range in size between 0.5 to 100 cc. However, the size of the syringe is not limiting with respect to the invention. Other sizes can also be used. In further aspects, the size of the needle may range between a 16 and 30 gauge needle. Like the syringe, needle size is not limiting with respect to the invention.

In certain aspects, a single bolus injection is given by intravenous infusion or by direct injection, using a syringe. This mode of administration may be desirable in surgical patients, if appropriate, such as patients having cardiac surgery, e.g., coronary artery bypass graft surgery and/or valve replacement surgery. In these patients, a single bolus infusion of fibrin clot can be administered. (Note that the amount of drug administered is based on the weight and condition of the patient and is determined by the skilled practitioner.) In other aspects, a single injection is given intramuscularly. Shorter or longer time periods of administration can be used, as determined to be appropriate by one of skill in this art.

In cases in which longer-term delivery of the composition is desirable, intermittent administration can be carried out. In these methods, a loading dose is administered, followed by either (i) a second loading dose and a maintenance dose (or doses), or (ii) a maintenance dose or doses, without a second loading dose, as determined to be appropriate by one of skill in this art.

In various aspects, to achieve further delivery of the composition in a patient, a maintenance dose (or doses) of the fibrin clot is administered. Maintenance doses can be administered at levels that are less than the loading dose(s), for example, at a level that is about ⅙ of the loading dose. Specific amounts to be administered in maintenance doses can be determined by a medical professional, with the goal that the composition comprising fibrin clot and cytokine is at least maintained at the target cite for a period of time. Of course, maintenance doses can be stopped at any point during this time frame, as determined to be appropriate by a medical professional.

In other aspects of the invention, delivery is made with a catheter. Delivery by catheter can be carried out by using products (for example, infusion pumps and tubing) that are widely available in the art. One criterion that is important to consider in selecting a catheter and/or tubing to use in these methods is the impact of the material of these products (or coatings on these products) on the composition comprising fibrin clot and cytokine. Additional catheter-related products that can be used in the methods of the invention can be identified by determining whether the material of the products alters the composition comprising fibrin clot and cytokine, under conditions consistent with those that are used in drug administration.

Dosing

The invention includes the use of various dosing parameters. In one aspect, the cytokine is dosed per ml of fibrin clot or per kilogram body weight of a subject in need thereof. In various aspects, a subject receives multiple doses or multiple instances of treatment. In certain aspects, it is possible to re-dose a subject for increased or prolonged effects (weeks, months, or even years into the future). In dosing, the fibrin clot displaces a set amount of volume in the muscle and/or surrounding areas. Therefore, it is important to monitor the volume of cytokine/clot that can be injected. In one aspect, the maximum amount of cytokine to be delivered per fibrin clot is dictated by the volume (size) of the fibrin clot components used as well as the area of treatment and the size of the subject. In some aspects, larger subjects tolerate larger volumes of fibrin clot, making increased dosing or volume of cytokine desirable.

One skilled in the art will appreciate that the appropriate dosage levels for treatment will thus vary depending, in part, upon the tissue site to which the composition comprising fibrin clot and cytokine is delivered, the indication for which the composition is being used, the route of administration, and the size (body weight, body surface or organ size) and condition (the age and general health) of the patient. Accordingly, the clinician may titer the dosage and modify the route of administration to obtain the optimal therapeutic effect.

In certain aspects, multiple clots are delivered. In other aspects of the invention, the fibrin clot comprising cytokine is administered at multiple time points. The frequency of clot administration depends upon multiple parameters. Typically, a clinician will administer the composition until a dosage is reached that achieves the desired effect. The composition may therefore be administered as a single dose, or as two or more doses (which may or may not contain the same amount of cytokine) over time. Further refinement of the appropriate dosage is routinely made by those of ordinary skill in the art and is within the ambit of tasks routinely performed by a clinician or a person of ordinary skill in the art. Appropriate dosages may be ascertained through use of appropriate dose response data.

Methods of Treatments and Uses

In aspects of the invention, the fibrin clot comprising cytokine is used in recruiting regenerative or reparative cells to a site in vivo for tissue regeneration or repair after tissue damage or loss resulting from disease or injury, including injury from surgery. In certain aspects, the fibrin clot comprising cytokine is used in enhancing vasculogenesis or inducing angiogenesis. The invention includes using the compositions described herein for any disease or condition which can benefit from the administration of a fibrin clot comprising cytokine to increase vascularization at a specific site in vivo. For example, tissue damage due to ischemia due to blood flow loss, lacerations, extremes of temperature, trauma, or metabolic or genetic disease, is one of many various conditions or diseases which can benefit from treatment with cytokine in a fibrin clot. Other diseases include cardiovascular disease, diabetes, autoimmune diseases, stroke, brain and/or spinal cord injury, burn injury, bone defects, renal ischemia, and macular degeneration. In another aspect, the compositions of the invention are used to treat an ischemic or a cirrhotic liver. In various aspects, the invention includes uses of a composition comprising fibrin clot and cytokine for the manufacture of a medicament for treating a localized site of injury or disease, for enhancing vascularization to a localized site, or for treating ischemia. In certain aspects, a composition of the invention is used to enhance vascularization of a transplanted organ or tissue. Organs that are transplanted include, without limitation, the heart, kidneys, liver, lungs, pancreas, intestine, and skin. Tissues include, without limitation, bones, tendons, cornea, heart valves, veins, and arms.

Kits

In a further aspect, the invention includes a kit for preparing the composition comprising fibrin clot and cytokine and administering it to a subject in need thereof. The composition comprising fibrin clot and cytokine may be advantageously provided in kit form including separately packaged amounts of fibrin sealant and thrombin. In another aspect, the kit may further comprise cytokine from another source. Alternatively, the kit can include an additional biological agent that can be delivered in the fibrin clot or in conjunction with the administration of the fibrin clot and a cytokine. In an exemplary embodiment of a kit, each component of the kit is packaged separately in sterile packaging or in packaging susceptible to sterilization. The biological agents, including the fibrinogen component, the thrombin component, or the cytokine, may be provided in a container such as a glass or plastic vial and may further be carried or suspended in a liquid storage medium suitable for maintaining the cytokine or other biological compounds. The kit may optionally further include one or more syringes, catheters or other delivery device(s) for introducing the fibrin clot into the subject. Kits may optionally further include one or more additional containers each storing a pharmaceutical agent that may be added to the fibrin clot. The kit further includes, for example, printed instructions for making and using the fibrin clot. All elements of the kit are provided together in suitable amounts in a box or other suitable packaging.

Each publication, patent application, patent, and other reference cited herein is incorporated by reference in its entirety to the extent that it is not inconsistent with the present disclosure.

It is understood that the embodiments described herein are for illustrative purposes only and that various modifications or changes in light thereof will be suggested to persons skilled in the art and are to be included within the spirit and purview of this application and scope of the appended claims.

### EXAMPLES

The invention is described in more detail with reference to the following non-limiting examples, which are offered to more fully illustrate the invention, but are not to be construed as limiting the scope thereof. Those of skill in the art will understand that the techniques described in these examples represent techniques described by the inventors to function well in the practice of the invention, and as such constitute preferred modes for the practice thereof. However, it should be appreciated that those of skill in the art should in light of the present disclosure, appreciate that many changes can be made in the specific methods that are disclosed and still obtain a like or similar result without departing from the spirit and scope of the invention.

### Example 1

### SDF-1 has Affinity for Tisseel® VHSD

The aim of this experiment was to measure the affinity of SDF-1 towards Tisseel® VHSD. The complex interaction between the fibrin sealant/clot component, Tisseel® VHSD, and recombinant human SDF-1α (rhSDF-1α) was examined in real time using surface plasmon resonance (SPR). In this assay, a gold sensor chip was coated with the fibrin sealant protein component (FC) of Tisseel® through covalent bonds between the lysine residues of FC and the chip. (The FC of Tisseel® includes a mixture of fibrinogen fibronectin, albumin, and other proteins.) The sensor chip was equilibrated with phosphate-buffered saline (PBS) at pH 7.4 by flowing the PBS over the sensor chip, and a baseline was established. PBS was replaced with an injection of five concentrations of rhSDF-1α (ranging from about 10 nM to about 250 nM) at 0.1 ml/minute to study the association and equilibrium phases of the binding. Recombinant hSDF-1α was passed over the sensor chip for seven minutes, before the injections were stopped and replaced with a flow of PBS at pH 7.4. The dissociation phases of the interactions were observed for an additional seven minutes. Regeneration between injections was not necessary, because rhSDF-1α auto-dissociated from the FC. After one concentration of rhSDF-1α was bound and released from the FC, another concentration was tested. A series of sensogram data was analyzed by fitting the data to binding interaction models using Prism 4.0 by Graphpad.

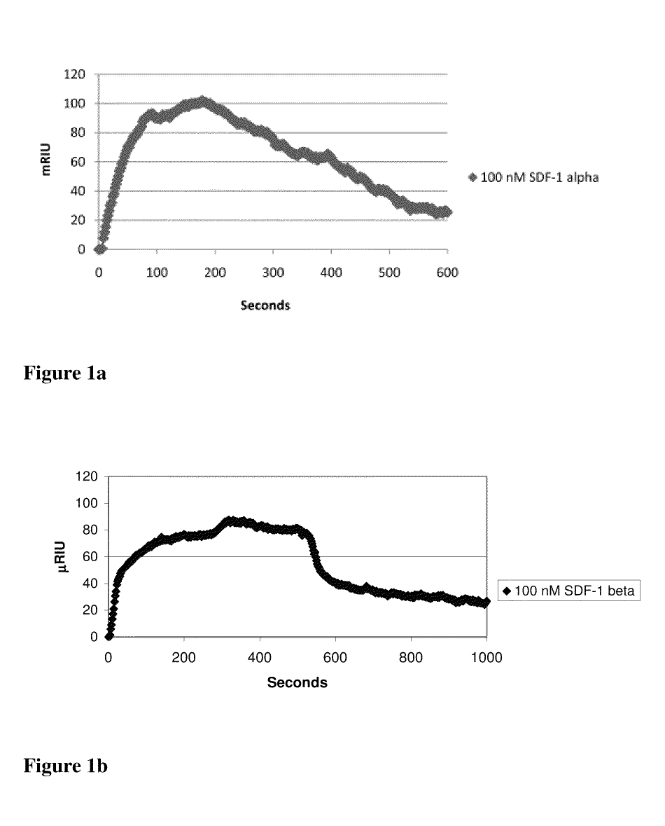

A resulting sensogram (FIG. 1a) displays the changes in the refractive index over time as a result of SDF-1α binding FC and indicates that SDF-1α has significant affinity for the FC of Tisseel® VHSD. Similar experiments were conducted using SPR to examine the interaction between recombinant human SDF-1β (rhSDF-1β) and FC. A new gold sensor chip was coated with FC, which was covalently bonded to the chip through accessible FC lysine residues. Like the experiment provided above, PBS was flowed over the sensor chip to equilibrate it and establish a stable baseline. PBS was replaced with an injection of five concentrations of rhSDF-1β (ranging from about 25 nM to about 500 nM) at 0.1 ml/minute to study the association and equilibrium phases of the binding. Recombinant hSDF-1β was passed over the sensor chip for seven minutes, before the injections were stopped and replaced with a flow of PBS at pH 7.4. The dissociation phases of the interactions were observed for an additional seven minutes. Regeneration between injections of rhSDF-1β was not necessary, because rhSDF-1β auto-dissociated from the FC. A series of sensogram data was analyzed by fitting the data to binding interaction models using Prism 4.0 by Graphpad.

A resulting sensogram (FIG. 1b) displays the changes in the refractive index over time as a result of SDF-1β binding FC and demonstrates that SDF-1β has significant affinity for the FC of Tisseel®VHSD.

### Example 2

### Preparation of Fibrin Clots to Determine Kinetics of SDF-1 Release In Vitro

The aim of the study was to determine the kinetics of SDF-1 release in vitro. Fibrin clots were prepared with 125 μl of FC diluted with fibrinogen dilution buffer (FDB) to 20 mg/ml fibrinogen and 4000 ng of SDF-1α. FDB contains 3000 KIU/ml aprotinin, 25 mM sodium citrate, 48 mM sodium chloride, 333 mM glycine and 15 g/L human serum albumin. SDF-1α and thrombin (125 μl of 4 U) were added to the FC to form the clots. Final concentrations in the clots were 10 mg/ml fibrinogen, 2000 ng of SDF-1α and 2 U of thrombin. The fibrin clots (gels) were allowed to polymerize at 25° C. before being washed with 250 μl of 0.1% human serum albumin in PBS. The gels were covered with 250 μl of 0.1% human serum albumin in PBS, and incubated at 25° C. for 1 to 7 days. The supernatant was removed from three fibrin clots on day one. The supernatants were stored at −20° C. and the clots were discarded. Supernatants were collected from three more gels on day two. This process was repeated with three new gels until the supernatants from the last gels were collected on day seven.

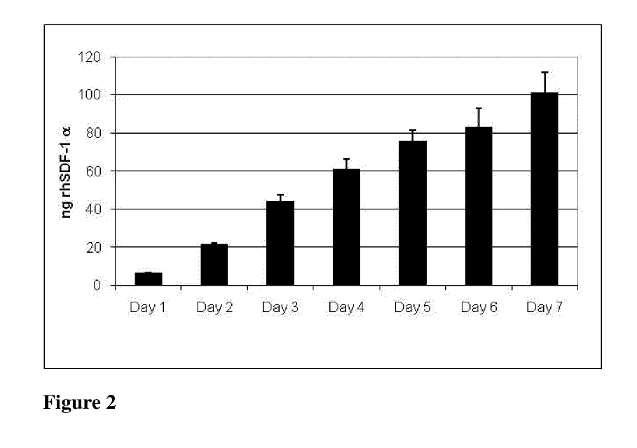

FIG. 2 shows the cumulative release of SDF-1α from fibrin clots from Tisseel® VHSD gels over seven days. SDF-1α concentrations were determined using an ELISA according to the manufacturer's instructions (R&D Systems, Minneapolis, Minn.). The average release of SDF-1α ranged from 6 ng (Day 1) to a cumulative release of 100 ng (Day 7), accounting for only 5% of the original amount of SDF-1α added to the fibrin clots (100 ng/2000 ng×100%). These results demonstrate that fibrin clots containing SDF-1 both release and retain SDF-1. In vivo, these release kinetics are likely to be impacted by degradation of Tisseel® VHSD which takes place over a 7-8 day period.

### Example 3

### CD45 and CD11B: Differentiation and Endothelial Tube Formation

The aim of the study was to study the angiogenic and differentiation abilities of CD34+ myeloid cells in the presence of endothelial cells. This study showed that CD34+ cells and their more differentiated progeny are both pro-angiogenic in this angiogenic assay. Thus, in certain circumstances, Tisseel-SDF-1 apparently demonstrates the ability to recruit both more mature (more differentiated) and less mature cells (less differentiated),

CD34+ cells were isolated from cord blood and evaluated using cytospin, flow cytometry analysis, and Matrigel in an in vitro tube formation assay. The freshly isolated CD34+ cells comprised about 91% blasts and about 85% CD34+ by cytospin and flow cytometry analysis, respectively. Additionally, the cells expressed approximately 33% triple positive staining for CD34/CD11b/CD45 markers. The cells' angiogenic capability was then evaluated using a Matrigel tube formation co-culture assay with human umbilical vein endothelial cells (HUVECs).

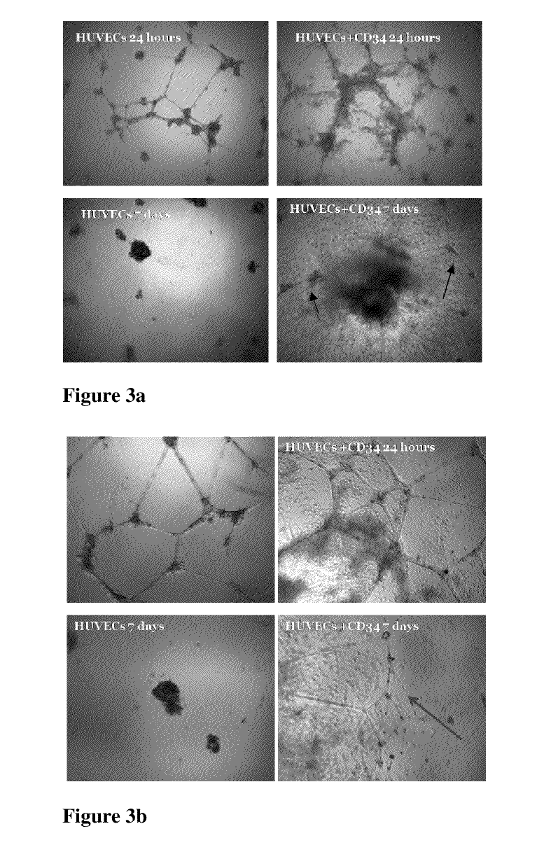

In the assay, HUVECs were seeded on Matrigel and grown for a period of 7 days without changing the media. Endothelial tube formation in the presence and absence of CD34+ cells was examined under phase microscopy, and images were taken at 24 hours and at 7 days (FIG. 3a). While HUVECs alone cannot maintain their tube structure, HUVECs in co-culture with CD34+ cells maintained tube structure for at least a week (see bottom right panel of FIG. 3a).

In addition to the angiogenesis (co-culture) assay described above, CD34+ cells in a Myelocult differentiation medium with granulocyte-macrophage colony-stimulating factor (GM-CSF) were grown for a period of 17 days. At the end of 17 days, the CD34+ cells became more differentiated: about 5-7% blasts, about 3-12% CD34+, and about 75% double positive for the CD11b/CD45 differentiation markers by cytospin and flow cytometry analysis. When this more differentiated population were evaluated for pro-angiogenic activity, this population also was able to sustain endothelial tube formation for at least a week in culture in a manner similar to less differentiated CD34+ cells. In both cases, both populations of myeloid cells were pro-angiogenic (see FIG. 3b). Without being bound by theory, it appears that Tisseel-SDF1's ability to recruit a more differentiated myeloid population using SDF-1 is likely to enhance neo-vascularization in situ.

### Example 4

### Recruitment and Retention of Reparative Cells In Vivo Using Tisseel SDF-1α and Tisseel-SCF-1

The aim of the study was to examine the recruitment and retention of reparative cells in vivo using Tisseel with and without SDF-1α. Tisseel clots, with (Tisseel-SDF-1α) and without SDF-1α (300 ng) (Tisseel), were implanted subcutaneously into murine hind limbs. Each mouse received a Tisseel clot implant in both legs, such that one leg received an implant of Tisseel and the other leg an implant of Tisseel-SDF-1α. Tisseel clots were recovered from the mice at day one, day two, day three, and day four after implantation. CD34+ cells that migrated into the clots were removed using a trypsin-EDTA digestion treatment. The number of cells that were recovered from the Tisseel implants that expressed Cd45, Cd11b, and Sca1 were quantified using fluorescence activated cell sorting (FACS).

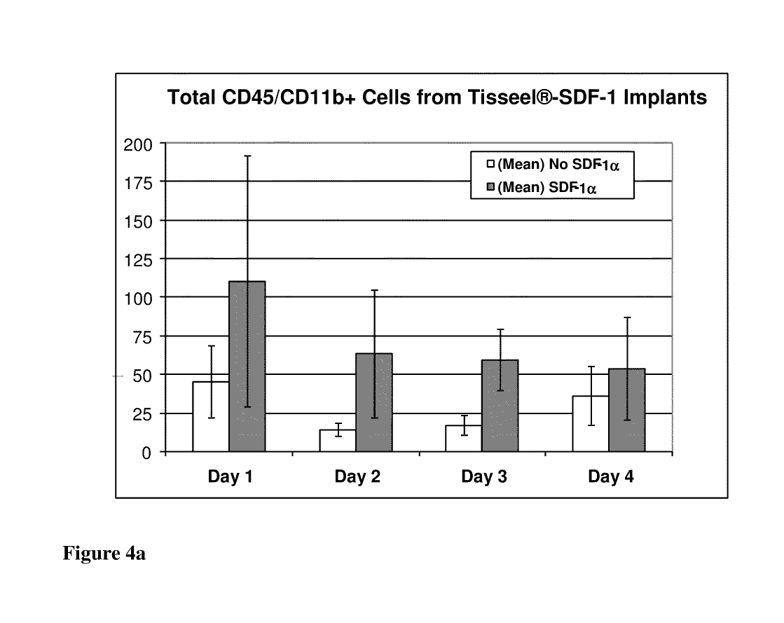

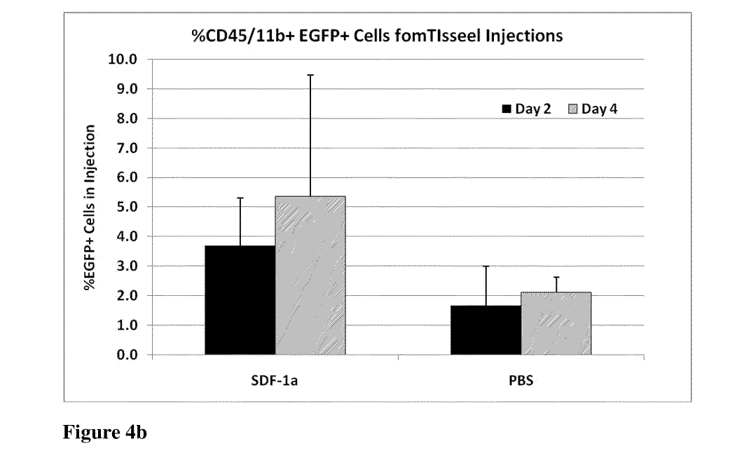

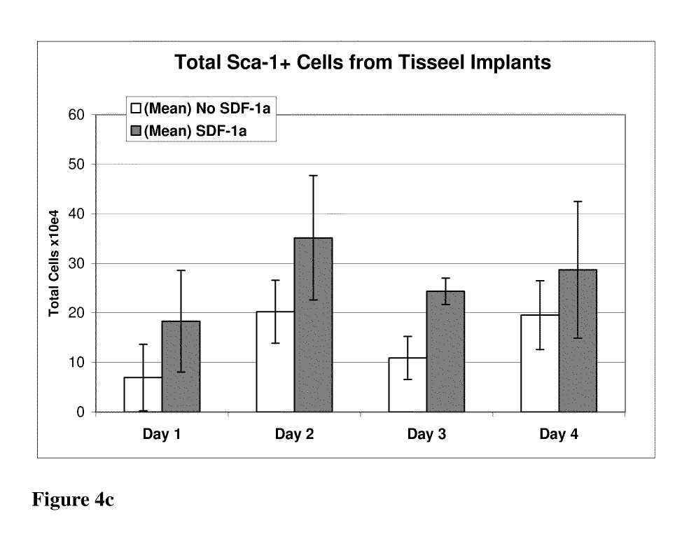

The results from this experiment (FIG. 4a) show cells that co-express CD45 and CD11b (CD45/11b+) were recruited to Tisseel-SDF-1 clots in greater numbers than to clots without SDF-1. Similarly, Tisseel-SDF-1 recruited greater numbers of cells that express the murine equivalent of CD34, Sca1 (see FIG. 4c). At all four time points, statistical analysis (ANOVA) indicates that the increase in cell number was statistically significant for the CD45/11b+ populations. Specifically, the value was 0.002 for the Sca1+ population and 0.003 for cells that co-express CD45 and CD11b (CD45/11b+).

Mouse hind limb studies were also carried out with Tisseel®+SDF-1α administered via intramuscular (i.m.) injections instead of implants. For these studies, enhanced green fluorescent protein (EGFP)+ cells were isolated from the femur and tibia bone marrow (BM) of donor mice (EGFP+ C57BL/6 transgenic mice (Jackson Labs). These transgenic mice constitutively express EGFP in all cells with the exception of hair and erythrocytes. The EGFP+ BM cells (5 million) were injected via tail vein injection into recipient non-EGFP+ mice. The recipient mice were sacrificed at day 2 and day 4 and the i.m. injection sites were removed, digested, and evaluated for the presence of EGFP+, CD45+, and CD11b+ cells. The results in FIG. 4b show that a greater percentage of EGFP+, CD45+, and CD11b+ cells were detected in the i.m. injection sites containing SDF-1α than in the i.m. injection sites without SDF-1α (Tisseel® control; PBS/0.1% BSA) at day 2 and day 4.

Mouse hind limb studies were also carried out for Tisseel®+ SCF administered via intramuscular (i.m.) injections instead of implants. For these studies, the mice received i.m. injections in one hind limb (50 μl of Tisseel® comprising SCF (300 ng)). Control mice received i.m. injections in one hind limb of Tisseel® (50 μl) without SCF. Following the hind limb injections, mice received EGFP bone marrow cells (5 million) as described herein above in the SDF-1α experiment. The recipient mice were sacrificed at day 2 and day 4 and the i.m. injection sites were removed, digested, and evaluated for the presence of EGFP+ and Sca-1+ cells. The results from this experiment show that a greater percentage of EGFP+Sca-1+ cells could be detected in the treated animals than the controls, indicating that SCF in Tisseel® helped recruit and retain more regenerative and/or reparative cells in vivo.

The invention has been described in terms of particular embodiments found or proposed to comprise specific modes for the practice of then invention. Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

1. A composition comprising a fibrin clot and a cytokine or a combination of cytokines.

2. The composition of claim 1, wherein the cytokine is selected from the group consisting of stromal derived factor-1 (SDF-1) and stem cell factor (SCF).

3. The composition of claim 2, wherein the cytokine is SDF-1.

4. The composition of claim 2, wherein the cytokine is SCF.

5. The composition of claim 1, wherein the combination of cytokines comprises stromal derived factor-1 (SDF-1) and stem cell factor (SCF).

6. The composition of claim 1, wherein the fibrin clot comprises any fibrin-based hemostat or sealant.

7. The composition of claim 6, wherein the fibrin clot is Tisseel® or Tisseel® VHSD.

8. The composition of claim 1, wherein the fibrin clot comprises fibrinogen at a final concentration from about 1 mg/ml to about 100 mg/ml and thrombin at a final concentration from about 0.5 IU/ml to about 250 IU/ml.

9. The composition of claim 8, wherein the fibrin clot comprises fibrinogen at a final concentration of about 10 mg/ml and thrombin at a final concentration of about 2 IU/ml.

10. The composition of any one of claims 1 further comprising phosphate buffer or a phosphate-buffered saline solution.

11. The composition of claim 2 comprising SDF-1 at a final concentration from about 1.0 ng/ml to about 50,000 ng/ml.

12. The composition of claim 11 comprising SDF-1 at a final concentration from about 10 ng/ml to about 5,000 ng/ml.

13. The composition of claim 2 comprising SCF at a final concentration from about 1.0 ng/ml to about 50,000 ng/ml.

14. The composition of claim 13 comprising SCF at a final concentration from about 10 ng/ml to about 5,000 ng/ml.

15. A method of preparing a composition comprising a fibrin clot and a cytokine or a combination of cytokines, the method comprising the step of mixing the cytokine or combination of cytokines with thrombin and adding the cytokine and thrombin to fibrinogen to form the fibrin clot composition.

16. The method of claim 15, wherein the cytokine is selected from the group consisting of stromal derived factor-1 (SDF-1) and stem cell factor (SCF).

17. A method for recruiting and retaining reparative cells to a localized site of injury or disease in a subject in need thereof, the method comprising the step of delivering the composition of any one of claims 1 to the site of injury or disease in an amount effective to recruit and retain reparative cells to the site of injury.

18. The method of claim 17, wherein the reparative cells are stem cells.

19. The method of claim 18, wherein the reparative cells are myeloid cells and their differentiated progeny.

20. The method of claim 17, wherein the reparative cells are positive for CD34 (CD34+), CD45 (CD45+), or CD11b (CD11b+).

21. The method of claim 17, wherein the reparative cells are positive for one or more of CD34 (CD34+), CD45 (CD45+), and CD11b (CD11b+).

22. A method for treating a localized site of injury or disease in a subject in need thereof, the method comprising the step of delivering the composition of any one of claims 1 to the site of injury or disease in an amount effective for treating the injury or disease.

23. A method of enhancing vascularization or inducing angiogenesis to a localized site of injury or disease in a subject in need thereof, the method comprising the step of delivering the composition of any one of claims 1 to the site of injury or disease in an amount effective for enhancing vascularization or inducing angiogenesis.

24. A method of treating ischemia in a subject, the method comprising the step of delivering a composition of any one of claims 1 to a site of ischemia in an amount effective to treat ischemia.

25. A kit for preparing the composition of any one of claims 1, the kit comprising:
(a) a first vial or first storage container comprising fibrinogen;
(b) a second vial or second storage container comprising thrombin; and
(c) a third vial or third storage container comprising a cytokine or a combination of cytokines, said kit optionally containing a phosphate buffer and instructions for use thereof.

26-30. (canceled)
